(19) United States
(12) Patent Application Publication
(10) Pub. No.: US 2018/0116732 A1
(43) Pub. Date: May 3, 2018

(54) Real-time Three Dimensional Display of Flexible Needles Using Augmented Reality

(71) Applicant: The Board of Trustees of the Leland Stanford Junior University, Palo Alto, CA (US)

(72) Inventors: Michael A. Lin, Stanford, CA (US); Jung Hwa Bae, East Palo Alto, CA (US); Subashini Srinivasan, Palo Alto, CA (US); Mark R. Cutkosky, Palo Alto, CA (US); Brian A. Hargreaves, Menlo Park, CA (US); Bruce L. Daniel, Stanford, CA (US)

(21) Appl. No.: 15/786,952

(22) Filed: Oct. 18, 2017

Related U.S. Application Data
(60) Provisional application No. 62/409,601, filed on Oct. 18, 2016.

(51) Int. Cl. A61B 34/20; A61B 90/00; A61B 17/34; G06T 19/00; G06F 3/01; G02B 27/01

(52) U.S. Cl. CPC A61B 34/20; A61B 90/361; A61B 17/3403; A61B 2034/2055; G06F 3/012; G06F 3/013; G02B 27/017; G06T 19/006

(57) ABSTRACT

A method of real-time 3D flexible needle tracking for image-guided surgery is provided that includes uploading, using a controller, a virtual model of a flexible needle to a calibrated 3D mixed reality headset, where the virtual model, establishing an initial position of a base of a flexible needle relative to a target under test, using the controller and the flexible needle, where the flexible needle includes sensors spanning from the base to along a length of the flexible needle, where the sensors communicate a position, a shape, and an orientation of the flexible needle to the controller, where the controller communicates the sensor positions to the calibrated 3D mixed-reality headset, and using the calibrated 3D mixed-reality headset to display in real-time a position and shape of the flexible needle relative to the target under test.

### CROSS-REFERENCE TO RELATED APPLICATIONS

This application claims priority from U.S. Provisional Patent Application 62/409,601 filed Oct. 18, 2016, which is incorporated herein by reference.

### STATEMENT OF GOVERNMENT SPONSORED SUPPORT

This invention was made with Government support under contract EB009055 awarded by the National Institutes of Health, under contract CA159992 awarded by the National Institutes of Health, and under contract CA009695 awarded by the National Institutes of Health. The Government has certain rights in the invention.

### FIELD OF THE INVENTION

The present invention generally relates to image-guided surgery. More specifically, the invention relates to image-guided surgery that incorporates mixed reality with a flexible biopsy needle.

### BACKGROUND OF THE INVENTION

When surgical biopsy needles are placed into a human body, the physician placing them is no longer able to see the shape, orientation and position of the needle tip because the body is opaque. There is a need to show, in real-time, the three-dimensional shape, orientation and position of a thin needle, or other similar flexible device, throughout the entire medical procedure, particularly when any portion of the device is hidden from direct view within the human body.

### SUMMARY OF THE INVENTION

To address the needs in the art, a method of real-time 3D flexible needle tracking for image-guided surgery is provided that includes uploading, using a controller, a virtual model of a flexible needle to a calibrated 3D mixed reality headset, where the virtual model, establishing an initial position of a base of a flexible needle relative to a target under test, using the controller and the flexible needle, where the flexible needle includes sensors spanning from the base to along a length of the flexible needle, where the sensors communicate a position, a shape, and an orientation of the flexible needle to the controller, where the controller communicates the sensor positions to the calibrated 3D mixed-reality headset, and using the calibrated 3D mixed-reality headset to display in real-time a position and shape of the flexible needle relative to the target under test.

In another aspect of the invention, the 3D mixed-reality headset is programmed to display an extension to the needle tip as a line such that it aids the user in perceiving the direction in which the needle tip is pointing.

According to one aspect of the invention, the calibrated 3D mixed reality headset includes a head-mounted optical see-through stereoscopic display configured to display 3D content in the surroundings of a user.

In another aspect of the invention, calibrated 3D mixed reality headset is configured for displaying 3D reconstructions of structures of the target under test when coupled to a medical imaging device selected from the group consisting of MRI, ultrasound and CT. The sensors communicate to the controller the reference frame of a patient which the controller can use to display these medical 3D images inside the patient such that the user performing a biopsy can steer the needle insertion based on these images.

According to a further aspect of the invention, the sensors include optical strain gauges.

In yet another aspect of the invention, the controller is configured to measure the relative position and orientation of the calibrated 3D mixed reality headset to the flexible needle and the target under test.

In another aspect of the invention, calibrating the 3D virtual reality headset includes using pupil-tracking cameras rigidly attached to the 3D mixed-reality headset and a computer vision algorithm to track eye positions, where a relative position between the eyes and the 3D virtual reality headset are provided.

### BRIEF DESCRIPTION OF THE DRAWINGS

FIG. 1 shows a perspective view of a physician performing a biopsy procedure in accordance with an embodiment of the present invention.

FIG. 2 shows a front view of a calibrated 3D mixed reality headset device. A controller communicates with a camera on the headset to detect the fiducial markers located on the needle and to calculate the position and orientation of the needle handle, according to one embodiment of the invention.

FIGS. 3A-3D show a biopsy needle under deflection and Fiber Bragg gratings in the needle sense changes in strain which are converted into needle deflection, according to one embodiment of the invention.

FIG. 4 shows a perspective view of the base of the biopsy needle and the placement of tracking markers for position/orientation sensing, according to one embodiment of the invention.

FIG. 5 shows a perspective view of a needle that is partially inserted into a patient. A reconstructed virtual needle is overlaid on the real needle matching on orientation and shape. A target location is also shown fixed to the patient, according to one embodiment of the invention.

FIGS. 6A-6B show the calibration between the user's eyes and the headset to establish a calibrated 3D mixed-reality headset, according to one aspect of the invention.

### DETAILED DESCRIPTION

The present invention provides a new real-time visual guidance modality to assist physicians during a needle insertion procedure. The invention is comprised by four major components:

1) A sensorized needle 10 or device that is instrumented with strain gauges that enable real-time measurements of the precise shape of that device. This may include needles sensorized with optical fiber Bragg gratings (FBG).

2) A controller operating a position tracking system that measures, in real-time, position and orientation of rigid parts of the needle such as the handle. The position tracking system may be based on electromagnetic trackers, stereoscopic optical system, room-anchored multi-camera triangulation system or computer vision system. In one embodiment, this component uses standard tag detection techniques.

3) Preoperative scans of the patient anatomy registered to the patient using fiducial markers. The scans may be gathered through medical imaging methods such as MRI, CT, PET, Fluoroscopy or Ultrasound.

4) A head-mounted computer and transparent display system, such as a calibrated 3D mixed-reality headset, that overlays a holographic reconstruction of the biopsy needle based on the real-time information (position, orientation, and shape) gathered from (2).

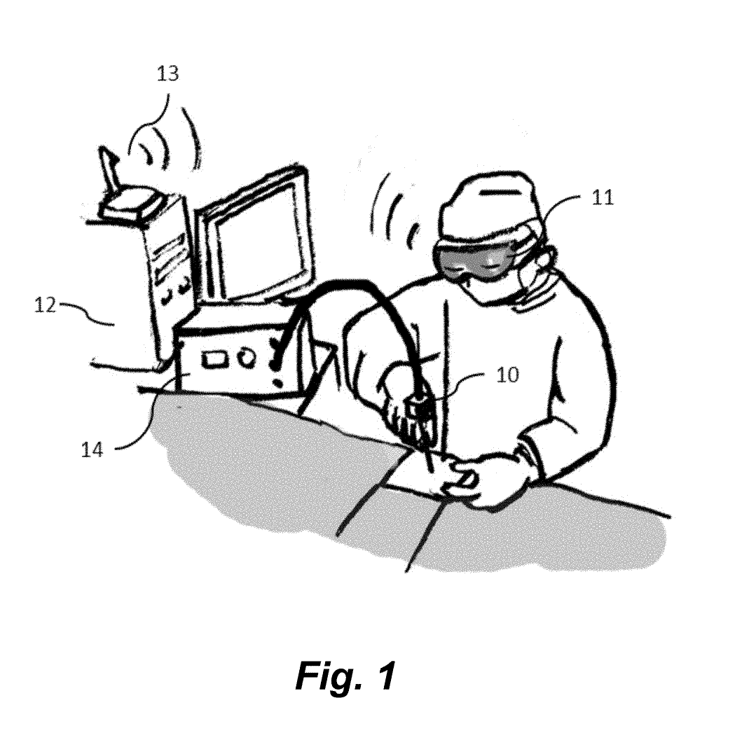

Referring to the drawings in detail, a physician using the visual guidance system during a needle biopsy procedure is shown in FIG. 1 according to the embodiment of the present invention. The physician performs a procedure wearing a calibrated 3D mixed-reality headset device 11, through which the physician can see holograms while having sight of the entire workspace due to the transparent display. The biopsy needle 10 is designed such that real-time position, orientation and shape can be extracted from it by a controller and used for displaying holographic aids to the physician.

This design includes, in addition to all the features of a traditional biopsy needle, two components:

(i) a plurality of optical strain gauges embedded along the needle stylet and

(ii) visual fiducials markers.

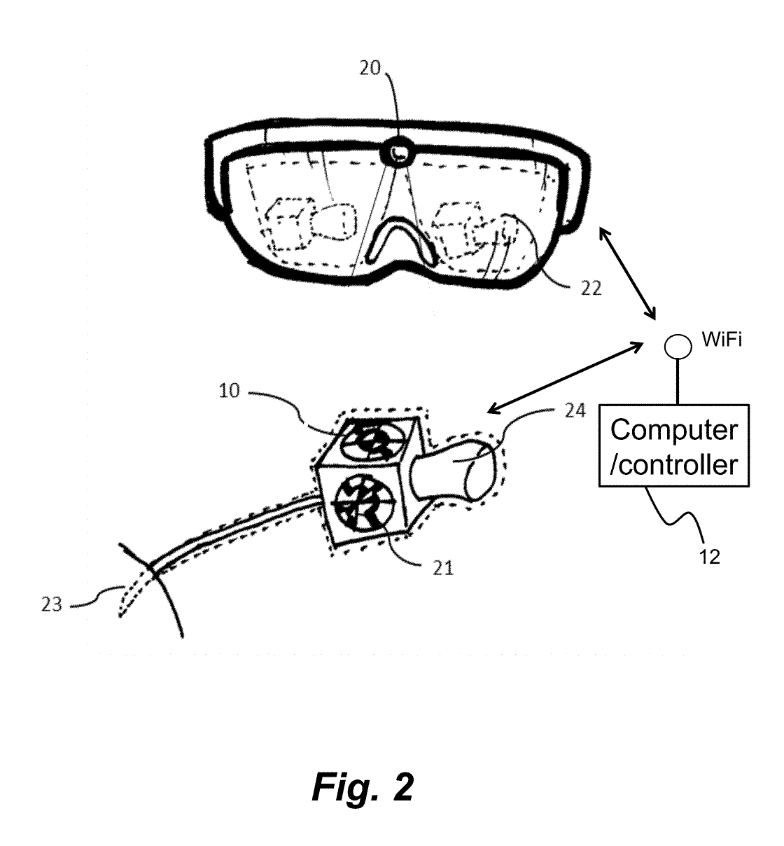

The signal from the optical strain gauges is measured using an optical interrogator 14 and the visual fiducial markers are measured with a digital camera 20 attached to the calibrated 3D mixed-reality headset 11. The measured data from the strain sensors are processed by a computer/controller 12 into needle shape data, which are then sent to the calibrated 3D mixed-reality headset 11 through a wireless connection fast enough so that the update rate is not visible (e.g. greater than 100 Hz). The calibrated 3D mixed-reality headset 11 uses the shape information with the position and orientation of the needle handle to display 22 a holographic representation of the needle 23 overlaid on the physical biopsy needle 10 (see FIG. 2).

As shown in FIG. 2, visual fiducial markers 21 may be fixed to rigid parts of the needle such as the handle 24 to track its pose (position and orientation). Using a digital camera 20 located on the calibrated 3D mixed-reality headset, the pose of these markers 21 can be detected with a computer vision program known in the art. Each marker has a unique pattern that the computer vision program associates with an identification number. The identification number of each marker, as well as its position and orientation, can be detected independently so if at least one marker 21 is successfully detected the position and orientation of the needle handle can be fully determined. The redundant placement of the markers 21 is important to guarantee that the needle 10 can be tracked by the digital camera 20 pointed at it from all perspectives.

Another variation of the method to perform needle position and orientation tracking is to use a magnetic tracking system. In this implementation, an electromagnetic transmitter would be placed within 0.5 meters distance from both the biopsy needle 10 and the calibrated 3D mixed-reality headset 11. A 6 degrees-of-freedom magnetic tracking sensor would be fixed to each the needle 10 and the calibrated 3D mixed-reality headset 11. Each magnetic tracking sensor is able to determine its own position and orientation given the presence of the electromagnetic field from the transmitter. The readings from both tracking sensors can be used to obtain the position and orientation of the needle 10 with respect to the calibrated 3D mixed-reality headset 11 through a simple translation matrix and a rotation matrix, respectively, calculated from the two readings.

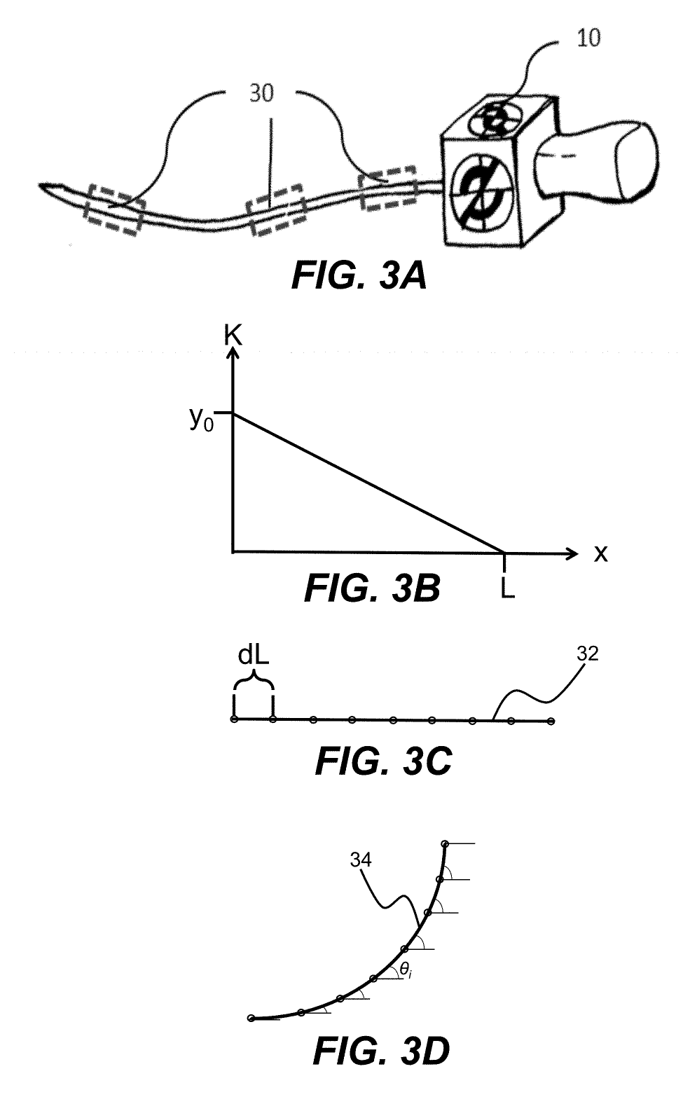

FIGS. 3A-3D show important aspects of the biopsy needle 10, where FIG. 3A shows a close up image of the biopsy needle 10 according to the embodiment of the present invention. The needle bending curvature sensing is achieved with a plurality of optical fibers with FBGs embedded in the needle coaxially. Each optical fiber has one grating located at each sensing region 30 and there may be multiple sensing regions along the needle. In one example, three sensing regions at distances of 53 mm, 125 mm and 149 mm from the needle handle 24 were used. An optical interrogator 14 coupled to the computer/controller 12 was used to read the wavelength shifts from the gratings, which correspond to changes in strain at the grating location. The relationship between strain data and needle curvature may be obtained through a linear fitting calibration. Beam theory describes the strain along the needle as linear given that the needle's interaction with tissue can be approximated as a tip load. A first order polynomial fit on the needle curvature was used at the three sensing locations 30 to obtain the curvature along the length of the needle 10. The computation of the needle curvature can be done in real-time by the computer/controller 12. This curvature information needs to be sent via a wireless connection to the holographic display 11. By using the polynomial representation of the needle curvature to stream the minimum amount of data in order to reduce effects of delay and update rate. For each rapid update rate only the coefficients of the curvature polynomial are transmitted, rather than a long data array containing the curvature value along various points of the needle. This is a key aspect of the invention, where the rapid data transfer corresponds to seamless image refresh rates.

On the compute side of the calibrated 3D mixed-reality headset 11, it is necessary to convert the curvature of the needle 10 to its deflection, and eventually build the mesh representation of the needle 10 using the deflection data. To obtain the deflection of the needle, constant length links connected through revolute joints were used. FIGS. 3B-3D show the process of using the needle curvature and a series of constant length links to obtain the needle deflection. Beginning with a needle 10 with no deflection 32 (see FIG. 3C) and data on the curvature K of a needle of length L. For each link i of length dL a curvature value is assigned that corresponds to the average curvature of that segment of the needle. The definition of needle curvature is K=dθ/dS where θ is the angle of each link from the horizontal line and S is the arc length. Since we use constant length links we can find the change in θ of each link through dθᵢ=Kᵢ*dL. To construct the deflection 34 of the needle (see FIG. 3D) each link was rotated such that θᵢ=dθᵢ+θᵢ₋₁.

Given the real-time needle shape data and the needle base 24 position and orientation data, the calibrated 3D mixed-reality headset 11 can construct a virtual needle 23 following these steps:

1) Use the data obtained from the visual fiducial system to display a virtual needle handle at the measured position and orientation of the physical needle handle.

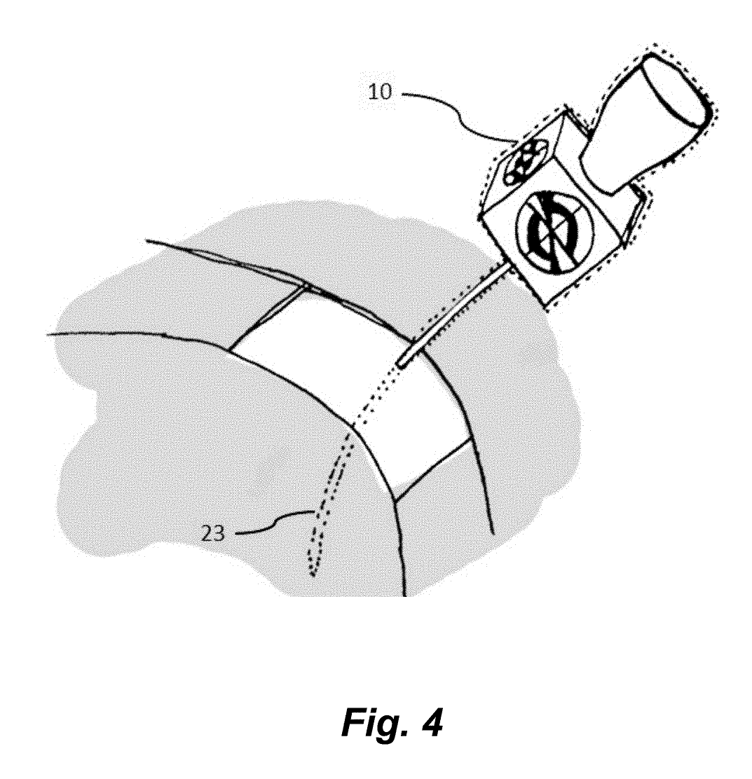

2) Use the polynomial representation of the shape of the needle to construct the shape of the needle shaft. This virtual needle can be constructed using procedural mesh generation to create a curved cylinder starting at the connection point on needle handle, following the needle shape data. As a result, every part of the holographic needle should be overlaying the physical needle. The wearer of the calibrated 3D mixed-reality headset 11 will be able to see the entire needle even when it has been partially occluded, as shown in FIG. 4.

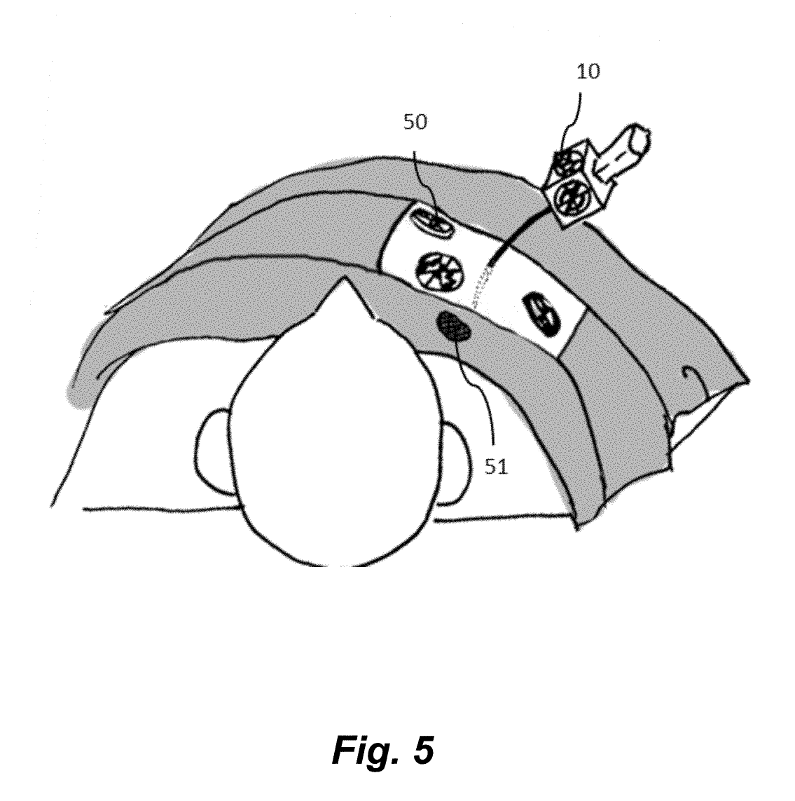

Another aspect of this invention is the integration of preoperative images into the image-guidance system. These preoperative images, which may be taken though MRI, CT, PET, fluoroscopy or ultrasound, reveal abnormalities, structures, anatomy and/or targets that the physician may want visualize in real-time while inserting the needle 10. These images may be registered to fiducial markers 50 fixed on the patient (see FIG. 5). The calibrated 3D mixed-reality headset 11 is configured to place 3D reconstructions of these critical structures 51 relative to the fiducial markers fixed on the patient 50, as shown in FIG. 5. The combined real-time display of the needle and preoperative data will help the physician guide the needle to its target while avoiding obstacles.

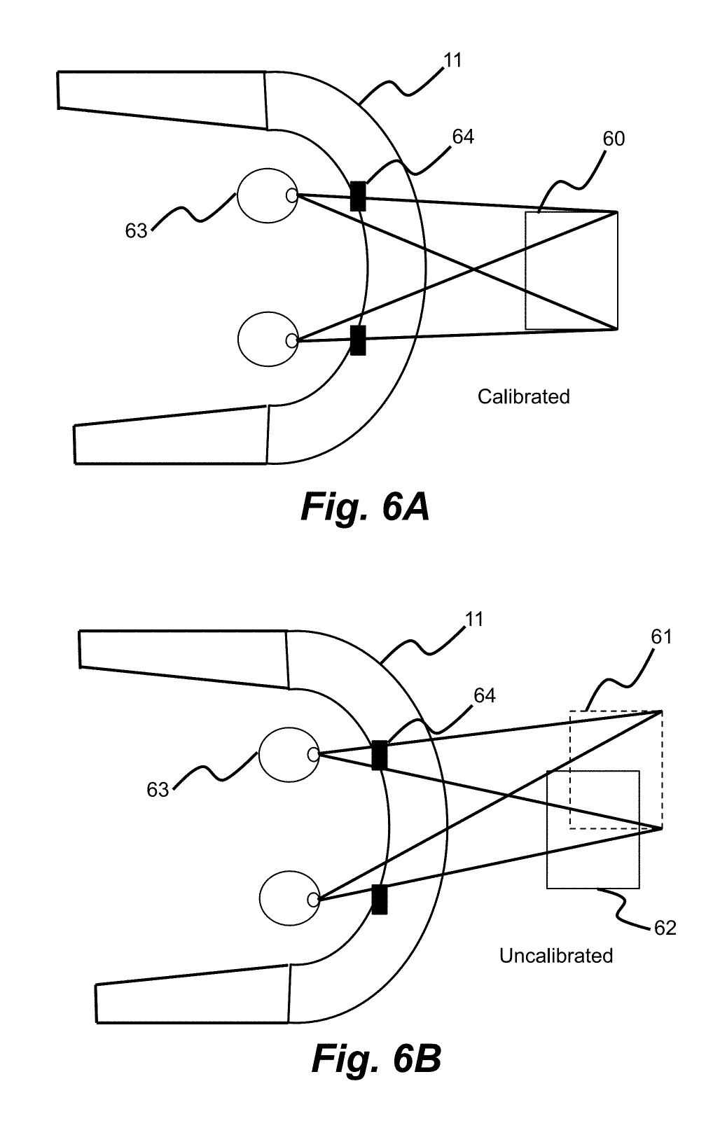

Since this augmented reality system is being used for visualizing objects at high position accuracy it is important to carefully perform the registration of the headset to objects and the patient. As shown in FIGS. 6A-6B, one important aspect is the calibration between the user's eyes and the calibrated 3D mixed-reality headset 11. Since the attachment of the users eyes 63 to the calibrated 3D mixed-reality headset 11 is not rigid it may be different for every time the user puts the display on. As shown in FIGS. 6A-6B, FIG. 6A shows the user wearing the calibrated 3D mixed-reality headset 11 and perceiving the virtual object aligned exactly to a physical object 60. FIG. 6B shows the same scenario, however the users eyes may be shifted off from where the uncalibrated 3D mixed-reality headset assumes the position of the user's eyes 63. This causes the user to perceive the virtual object 61 at a different location from the physical object 62. There are two solutions to calibrate for this misalignment: 1) use pupil-tracking cameras 64 which are miniature cameras rigidly attached to the display and use computer vision algorithms to track the eyes position and provide the relative position between the user's eyes and the mixed-reality display or 2) use a task-based alignment such as Single Point Active Alignment Method where the user manually aligns the virtual content to physical objects as it is perceived through the display to establish the calibrated 3D mixed-reality headset 11. The alignment done in method 2 is good until the headset moves with respect to the user's head, then the task-based alignment would need to be performed again.

In addition to the system's capability of superimposing a virtual needle on a real needle, the system can be programmed to extend the virtual needle tip as a line. Since the deflection of the needle is already known to the display device, this can be easily done by taking the 3D position and slope of the tip and drawing a 3D line extending out from the tip. Displaying this needle tip extension may help the physician better perceive the insertion angle of the needle, which may result in more accurate needle placement.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art.

All such variations are considered to be within the scope and spirit of the present invention as defined by the following claims and their legal equivalents.

What is claimed:

1) A method of real-time 3D flexible needle tracking for image-guided surgery, comprising:
a) uploading, using a controller, a virtual model of a flexible needle to a calibrated 3D mixed reality headset, wherein said virtual model;
b) establishing an initial position of a base of a flexible needle relative to a target under test, using said controller and said flexible needle, wherein said flexible needle comprises sensors spanning from said base to along a length of said flexible needle, wherein said sensors communicate a position, a shape, and an orientation of said flexible needle to said controller, wherein said controller communicates said sensor positions to said calibrated 3D mixed-reality headset; and
c) using said calibrated 3D mixed-reality headset to display in real-time a position and shape of said flexible needle relative to said target under test.

2) The method according to claim 1, wherein said 3D mixed-reality headset is programmed to display an extension to a tip of said flexible needle as a line, wherein said displayed line aids in visualizing a direction in which said needle tip is pointing.

3) The method according to claim 1, wherein said calibrated 3D mixed reality headset comprises a head-mounted optical see-through stereoscopic display configured to display 3D content in the surroundings of a user.

4) The method according to claim 1, wherein said calibrated 3D mixed reality headset is configured for displaying 3D reconstructions of structures of said target under test when coupled to a medical imaging device selected from the group consisting of MRI, ultrasound and CT.

5) The method according to claim 4, wherein said sensors communicate a reference frame of a patient to said controller, wherein said controller outputs instructions to display said 3D reconstructed structures inside said patient, wherein a user performing a biopsy is enabled to steer a needle insertion according to said displayed 3D reconstructed structures inside said patient.

6) The method according to claim 1, wherein said sensors comprise optical strain gauges.

7) The method according to claim 1, wherein said controller is configured to measure the relative position and orientation of said 3D virtual reality headset to said flexible needle and said target under test.

8) The method according to claim 1, wherein calibrating said 3D mixed reality headset comprises using pupil-tracking cameras rigidly attached to said 3D mixed-reality headset and a computer vision algorithm to track eye positions, wherein a relative position between said eyes and said 3D virtual reality headset are provided.
